(19) United States
(12) Patent Application Publication
(10) Pub. No.: US 2010/0204775 A1
(43) Pub. Date: Aug. 12, 2010

(54) Tissue Synthetic- Biomaterial Hybrid Medical Devices

(76) Inventor: Tarun John Edwin, Chandler, AZ (US)

Correspondence Address: COLLEN IP, THE HOLYOKE MANHATTAN BUILDING, 80 SOUTH HIGHLAND AVENUE, OSSINING, NY, 10562, US

(21) Appl. No.: 12/301,879

(22) PCT Filed: May 19, 2007
(86) PCT No.: PCT/US07/69318

§ 371 Date: Dec. 29, 2008

Related U.S. Application Data
(60) Provisional application No. 60/802,720, filed on May 22, 2006.

(51) Int. Cl. A61F 2/06; A61F 2/82

(52) U.S. Cl. 623/1.13; 623/1.42

(57) ABSTRACT

A hybrid medical device is described having at least one synthetic biomaterial, and at least one treated biological tissue suitable for implantation attached to the biomaterial wherein the tissue provides a blood contact surface and the biomaterial provides structural support. The tissue and biomaterial are attached using a polymer and the polymer is chemically or mechanically attached to the tissue. The device may further include pharmaceutical compounds for delivery over time and radiopaque compounds for fluoroscopic visualization. In some devices the tissue may be treated to degrade over time or the tissue only partially degrades over time. The biomaterial may be polytetrafluoroethylene (PTFE). In one configuration the device is configured for use as a heart valve.

### CROSS-REFERENCE TO RELATED APPLICATIONS

The present PCT patent application claims priority benefit of the U.S. provisional application for patent 60/802,720 filed on May 22, 2006 under 35 U.S.C. 119(e). The contents of this related provisional application are incorporated herein by reference for all purposes.

### FIELD OF THE INVENTION

The present invention relates to methods and compositions for the production and use of medical devices comprised of both tissue and synthetic biomaterial components.

### BACKGROUND OF THE INVENTION

Technological progress in biomaterials for medical devices has been relatively slow in the last 10 years. This is evidenced by the predominant and continued use of materials discovered over 30 years ago, such as polyester (polyethylene terephthalate) and PTFE (polytetrafluoroethylene) which to this day are still the dominant materials in products like vascular grafts, patches, and stent grafts.

In spite of its abundance, the use of animal tissue for medical or surgical use has been minimal, mainly due to ineffective processing methods required to render it useable for clinical applications. Most fixation methods that are required to stabilize the tissue so that it does not degrade in-vivo make use of aldehydes or similar chemicals that cross-link the protein, typically collagen. Although this treatment prevents degradation, the side effects include material weakness, toxicity, inflammation, calcification, and inferior handling properties. In spite of this however, there are many clinical uses of processed biological tissues in the literature. Commercial products include vascular patches, AV access grafts, wound dressings, and elements of heart valves. However it should be noted that due to their deficiencies, these products do not have a large share of the market and are typically niche products.

The gold standard for most vascular repair or bypass surgery is autogenous saphenous vein, harvested from the patient's own body. It is not possible for this material to be rejected by the body, and of course it handles well and performs excellently even with long term use, such as coronary bypass. Other uses of saphenous vein include below knee bypass to re-vascularize the leg to treat patients with peripheral vascular disease (PVD) and patching to assist closure and prevent stroke after carotid endarterectomy. However, it should be noted that often the vein harvested is not strong enough for the intended application, and cases of dissection or blowout have been reported.

Synthetic materials do not perform well in coronary bypass, and are neither used nor approved for this application. This is because intimal hyperplasia or a gradual stenosis of the lumen, results as the body rejects this foreign substance. Synthetic materials also never completely endothelialize or heal, in humans and so are a constant source of irritation to the blood stream, which continues to attack this obvious foreign body often resulting in blood clotting, or inflammation in the case of polyester grafts. There is a huge unmet need for an off-the-shelf biomaterial that can be used for coronary bypass since often saphenous or other vein is not available either because it has already been used up, or is friable and/or diseased. In addition, the surgical time that is required in order to harvest the vein is significant and the additional patient recovery time required is often detrimental to the patient. Regarding cost, the additional surgery time is expensive and the patient recovery time adds up to higher hospitalization costs.

Similarly, PTFE used as a bypass graft in the leg, does not work well below knee with patency rates less than 50% after two years, often due to distal graft intimal hyperplasia. PTFE used in above knee bypasses works better, but even this has inferior long-term patency rates when compared to saphenous vein, which is again the benchmark for performance.

In North America, PTFE is also the dominant material used in arterio-venous shunts (AV access) or for dialysis access in patients with end stage kidney disease. However, 50% of these grafts require intervention by the first year of implant. The reason for this is again due to venous end intimal hyperplasia, which reduces graft blood flow rates, increases intra-graft blood pressure, and can lead to clotting and occlusion of the graft. Native fistulas comprising the patients own arteries and veins are preferred for long term durability, however a large percentage of these fistulas do not “mature” (i.e. develop appropriately) and cannot be used for dialysis, forcing the patient to remain dependent on a temporary catheter system which is fraught with infection, clotting, and fibrin occlusion problems. An off-the-shelf biomaterial that can be used for AV access that handles and performs like vein would be a boon to the patient, nephrologist, and surgeon.

The 90's brought the introduction of minimally invasive technology. This is typically catheter based, so that a small cut-down incision for catheter entry is required instead of a large surgical exposure. An example of a minimally invasive device is a catheter-loaded compressed stent that is introduced into the arterial system via a cut-down into a small superficial artery, to treat an arterial stenosis, or narrowing. The radiopaque catheter is typically tracked over a guide wire using fluoroscopy for guidance. Once in place at the site of the stenosis, the catheter is pulled back allowing the stent to expand and buttress the artery pushing the plaque or occlusive material against the arterial wall, thereby allowing the normal flow of blood to resume. However, since stents are typically mesh-like structures, (like “chicken-wire”) there is always a possibility that the plaque can grow back through the walls of the stent causing restenosis. In order to remedy this, a thin covering made out of a material such as PTFE is placed on the outside, inside, or both surfaces of the stent to block the infiltration of plaque as described in U.S. Pat. No. 5,749,880 to Banas et al. However, since the stent-covering is typically a synthetic material, there are again issues with the blood components recognizing and re-acting to this foreign body. There is again an unmet need for a stent graft that utilizes tissue as the blood contact surface thereby minimizing or eliminating the chance of attack by blood-borne components. If the tissue stent-covering used allows endothelialization, the overall patency and longevity of stent grafts will be greatly enhanced. In another prior art, U.S. Pat. No. 6,110,212 issued to Gregory et. al, there is disclosed tissue preparation methods with the use of elastin and elastin based materials that are then formed into sheets and used for repair or replacement of human tissue in conjunction with tissue welding techniques. Nevertheless, this is limited to elastin based materials, whereby it does not utilize the properties of tissue degradation and does not teach the use of a third material to bond tissue to biomaterials. Additionally, the prior art does not comprise of expanded PTFE, and does not identify the importance of porosity for applications where cellular in growth is a necessity for healing. Further, there is not provided a functional stent graft.

Stroke, caused by inadequate supply of blood to the brain is the most common cause of neurological disability. Thirty percent of stroke sufferers are permanently disabled. A narrowing of the carotid arteries that supply blood to the brain is the cause of eighty percent of strokes. If the stenosis or narrowing is more than 70% of the original lumen however, a stroke can be prevented surgically by removing the plaque that is creating this blockage. A surgical incision into the carotid artery is made, and the plaque is cut out and removed. The artery is then sutured back together, but usually a patch over the incision is required so that the arterial lumen is not narrowed, and PTFE or polyester patches are used as the material of choice for closing the artery. Although harvested vein patches are sometimes used as well, they are often too weak for the application and have been known to dilate and burst under the high blood pressures seen in the carotid artery. Once again, the blood contact surface of the foreign material can be problematic. An off-the-shelf reinforced tissue patch would be extremely beneficial to the patient as well as to the surgeon.

Nearly 5 million people in the U.S. are candidates for hernia surgery, of which more than 700,000 will seek treatment every year. Hernia repair procedures have a re-intervention rate of 10-20% mostly caused by infection and post-operative adhesions. There is an unmet need for a product that will be anti-infective by healing rapidly into the tissue bed, and will resist adhesions when interfaced with the abdominal contents or bowels. Similar to the vascular patch above, a tissue biomaterial hybrid device will serve this need.

It is evident from the descriptions above that synthetic biomaterials such as PTFE/polyester as well as processed animal/human tissue products; have their drawbacks. The former is not completely biocompatible and therefore is not the first choice of material and the latter is often too weak, cytotoxic, or lacking the proper physical properties to be used in all applications. Neither can the latter be used for drug delivery, or rendered radiopaque.

In view of the foregoing, there is a need for improved biomaterials for medical devices.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is longitudinal cross sectional view of an exemplary stent graft or intra-luminally supported graft in accordance with an embodiment of the present invention;

FIG. 2 is an expanded three dimensional view of the exemplary stent graft of FIG. 1;

FIG. 3 is a radial cross sectional view of the exemplary stent graft of FIG. 1 compressed into a delivery catheter;

FIG. 4 is a perspective view of an exemplary vascular patch in accordance with an embodiment of the present invention;

FIG. 5 depicts an exemplary arterio-venous (AV) access graft in accordance with an embodiment of the present invention;

FIG. 6 illustrates the exemplary use of a space between the tissue and biomaterial surfaces to create a cannulation region for the AV access shunt in FIG. 5;

FIG. 7 illustrates an exemplary bypass graft in accordance with an embodiment of the present invention; and

FIG. 8 depicts a longitudinal cross sectional view of the exemplary bypass graft of FIG. 7

Unless otherwise indicated illustrations in the figures are not necessarily drawn to scale.

### SUMMARY OF THE INVENTION

To achieve the foregoing and other objects and in accordance with the purpose of the invention, tissue synthetic-biomaterial hybrid medical devices are presented.

In one embodiment, a hybrid medical device is presented. The device includes at least one synthetic biomaterial, and at least one treated biological tissue suitable for implantation attached to the biomaterial wherein the tissue provides a blood contact surface and the biomaterial provides structural support. In other embodiments the tissue and biomaterial are attached using a polymer and the polymer is chemically or mechanically attached to the tissue. Another embodiment further includes pharmaceutical compounds for delivery over time. Another embodiment further includes radiopaque compounds for fluoroscopic visualization. In other embodiments, the tissue degrades completely over time or the tissue only partially degrades over time. In still another embodiment, the biomaterial is polytetrafluoroethylene (PTFE). In a further embodiment, the PTFE is expanded and porous in nature. In a further embodiment, the device is configured for use as a heart valve.

In another embodiment a stent graft for opening a lumen of a blood vessel is presented. The stent graft includes a stent support structure with struts, an internal surface and an external surface, at least one treated biological tissue suitable for implantation disposed on the internal surface to provide a luminal surface for the blood vessel, and at least one synthetic biomaterial disposed on the external surface wherein the tissue and the biomaterial are attached through openings between the struts and the stent graft is compressible for delivery into the blood vessel via a catheter. In other embodiments, the stent support structure includes shape memory properties and the stent support structure includes treated biological tissue. In further embodiments, the stent support structure is metal or plastic, either permanent or temporary. In still another embodiment, the synthetic biomaterial is a low friction lubricious material for ease in loading and deployment from the catheter. In yet another embodiment, the tissue material is biostable and supports endothelialization and healing. In a further embodiment, the tissue material is completely or partially degradable over time. A further embodiment includes radiopaque materials or markers. In another embodiment, the tissue and biomaterial are attached using a polymer. Another embodiment further includes pharmaceutical compounds for delivery over time. In a further embodiment, the tissue has a high coefficient of radial expansion allowing the use of an angioplasty balloon to assist the stent graft to expand radially. In still another embodiment, the biomaterial is polytetrafluoroethylene (PTFE). In a further embodiment, the PTFE is expanded and porous in nature.

In another embodiment, a vascular patch is presented. The vascular patch includes a synthetic biomaterial and a treated biological tissue suitable for implantation and attached to the biomaterial wherein the tissue provides a blood contact surface, the biomaterial provides structural support, the patch is suitable for suturing and the patch can be trimmed to fit. In further embodiments the tissue is attached to the biomaterial using a polymer and the polymer provides additional resistance to suture-hole bleeding. Yet another embodiment further includes pharmaceutical compounds. In still another embodiment, the biomaterial is polytetrafluoroethylene (PTFE). In a further embodiment, the PTFE is expanded and porous in nature.

In another embodiment an arterio-venous (AV) access graft is presented. The AV access graft includes a continuous treated biological tissue suitable for implantation forming a luminal blood-contact layer and a synthetic biomaterial attached to the tissue forming an abluminal layer providing structural support to the tissue when the graft is sutured between a vein and an artery. Further embodiments include a cannulation region between the tissue and the biomaterial and a sealant disposed in the cannulation region for sealing a hole produced by a dialysis needle. In another embodiment, the sealant includes pharmaceutical compounds. In still another embodiment, the biomaterial is porous and at least some of the pores are filled with a gelatin material to encourage tissue incorporation. In further embodiments the biomaterial is lubricious for ease in tunneling and is polytetrafluoroethylene (PTFE).

In another embodiment, a bypass graft is presented. The bypass graft includes a tubular synthetic biomaterial of suitable dimensions for use in bypassing a blood vessel and a treated biological tissue suitable for implantation attached to a distal end of the biomaterial wherein the tissue mitigates effects of intimal hyperplasia. A further embodiment includes treated biological tissue suitable for implantation attached to a proximal end of the biomaterial wherein the tissue mitigates effects of intimal hyperplasia. Another embodiment further includes spiral or ringed beading on the abluminal surface of the biomaterial to provide kink and crush resistance. A further embodiment includes treated biological tissue suitable for implantation used as the luminal surface of the graft. In still another embodiment, the tissue is attached at the distal and proximal ends by suturing. In yet another embodiment the biomaterial is polytetrafluoroethylene (PTFE). In a further embodiment, the PTFE is expanded and porous in nature.

In another embodiment, a hybrid medical device is presented. The device includes means for providing at least one synthetic biomaterial, means for providing at least one treated biological tissue suitable for implantation and means for attaching biomaterial to the tissue wherein the tissue provides a blood contact surface and the biomaterial provides structural support as well as a matrix for cellular infiltration or tissue ingrowth A further embodiment includes means for providing pharmaceutical compounds. Still another embodiment includes for providing radiopaque compounds.

Other features, advantages, and object of the present invention will become more apparent and be more readily understood from the following detailed description, which should be read in conjunction with the accompanying drawings.

### DETAILED DESCRIPTION OF THE PREFERRED EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments.

Due to the shortcomings of both tissue and synthetic biomaterials, it is an aspect of the present invention to provide tissue synthetic-biomaterial hybrids that solve the problems of these individual components themselves. WIPO patent application #WO/2006/026325 to Pathak and Thigle describe novel and unique methods of processing animal and human tissue so that the current clinical issues are solved. Issued USPTO patents such as U.S. Pat. Nos. 5,749,880 to Banas et al. and 6,797,217 to McCrea et al. propose methods of attaching synthetic biomaterials to stents for various applications. Issued U.S. Pat. No. 5,100,422 to Berguer describes a vascular patch made out of several synthetic materials, and U.S. Pat. No. 6,517,571 to Brauker describes combining two PTFE layers to serve as a vascular patch. The present invention takes all of these concepts several steps further by proposing the combination of tissue and biomaterial in order to solve every single deficiency one might have alone.

WO/2006/026325 describes unique processing of animal tissue that eliminates rejection, inflammation, and calcification, and that can also be configured to attach drug-loaded polymeric materials such as polyethylene oxide (PEO) for drug delivery. The PEO can also serve as a mechanical adhesive as it can be applied in liquid form and then hardened. It also covers the treatment of this tissue for complete, partial, or no degradation in-vivo. In addition, it supplies the ability to create shape memory properties for the tissue so that it can be used as a stent-like structure. The tissue, if properly treated, is eligible for endothelialization, healing, and incorporation especially when used in vascular applications as the collagen tissue matrix has receptor sites for cell attachment and growth. Furthermore, when treated in this way, the tissue retains its natural feel and handling properties keeping it akin to the gold standard, autogenous saphenous vein. The inventors also describe the attachment of radiopaque compounds to the tissue, which can be either temporary or permanently bound.

It is an aspect of the invention to use the appropriately treated tissue in combination with one or more known biomaterials to solve the deficiencies of several different biomedical devices, only a few of which are mentioned here by way of example. Appropriate treatment of the tissue as described in WO/2006/026325 is preferred, however other methods of treating animal tissue may also be acceptable, such as described by Freeman et al. in U.S. Pat. No. 4,798,611. The use of glutaraldehyde to cross link the tissue is described here as well as gamma sterilization to reduce immunogenicity and to enhance handling characteristics. Yet another approach to the treatment of tissue for clinical use is described in U.S. Pat. No. 6,132,986 to Pathak et. al. where tissues are exposed to di or polyfunctional acids. Another tissue preparation that may be used if biodegradable tissue is desired as described in U.S. Pat. No. 6,652,594 to Francis et al. in which the material is treated by alkylating its primary amine groups to reduce antigenicity, but permitting its use in-vivo without crosslinking Pathak et al. also describes a non-glutaraldehyde crosslinking technique in U.S. Pat. No. 6,596,471 using a bis-maleimide compound. Appropriate treatment of tissue in this context refers to tissue processed in such a way that it retains its strength, natural feel and handling properties; is not cytotoxic or inflammatory, and is biostable or with controllable degradable properties. Other techniques for appropriate treatment of the tissue will be readily apparent to those skilled in the art in light of the teachings of the present invention.

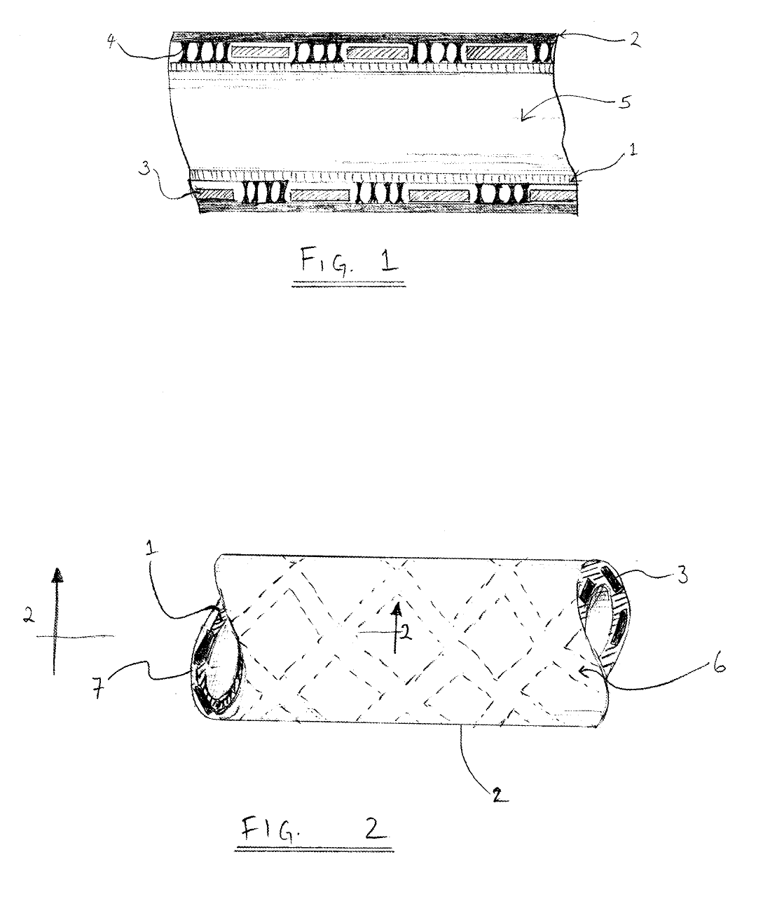

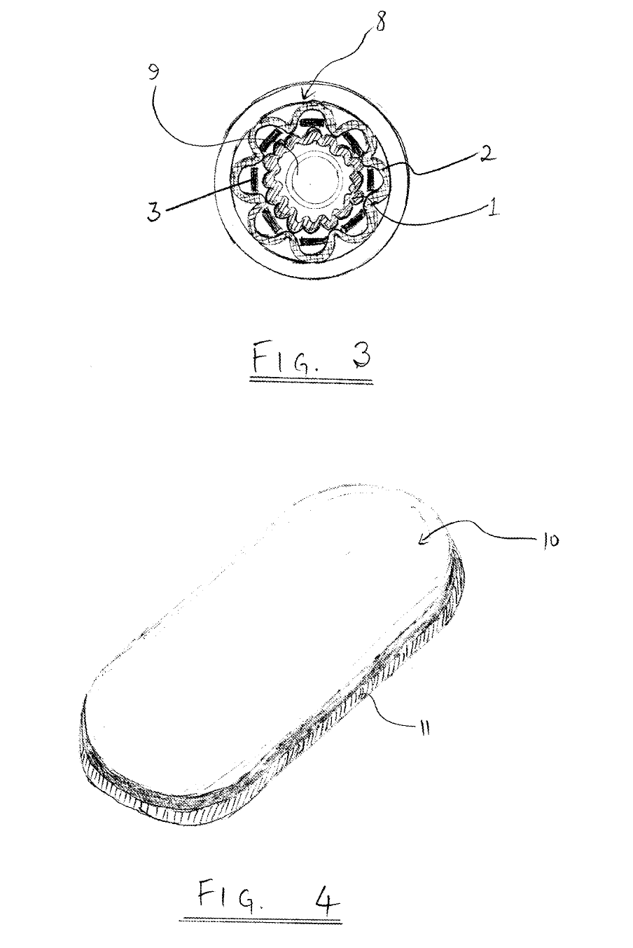

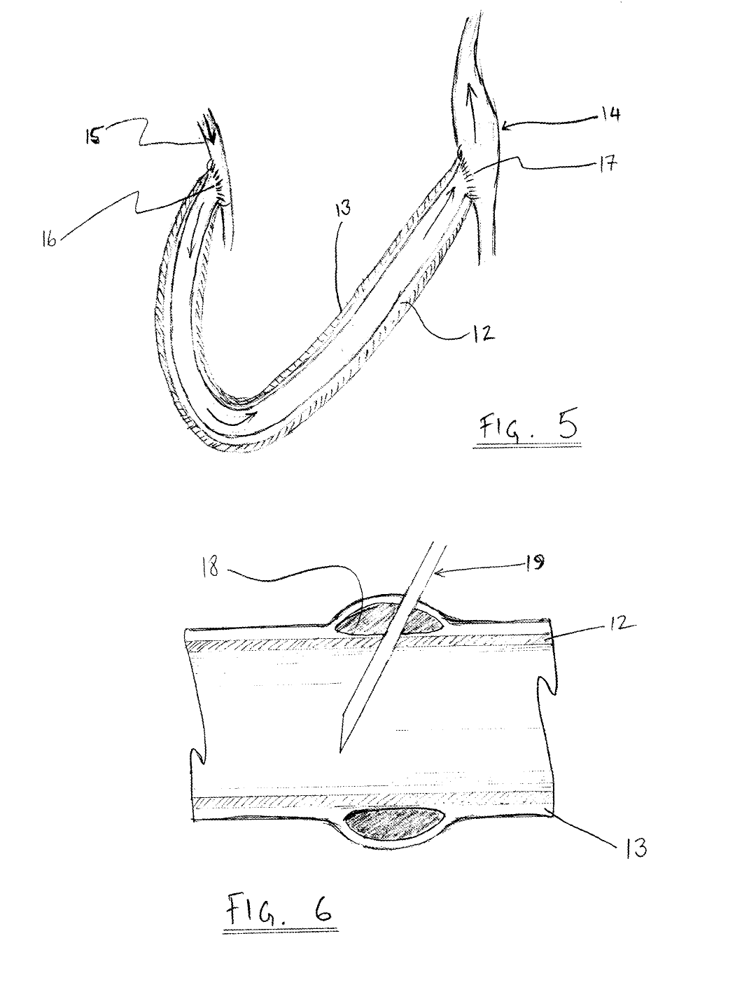

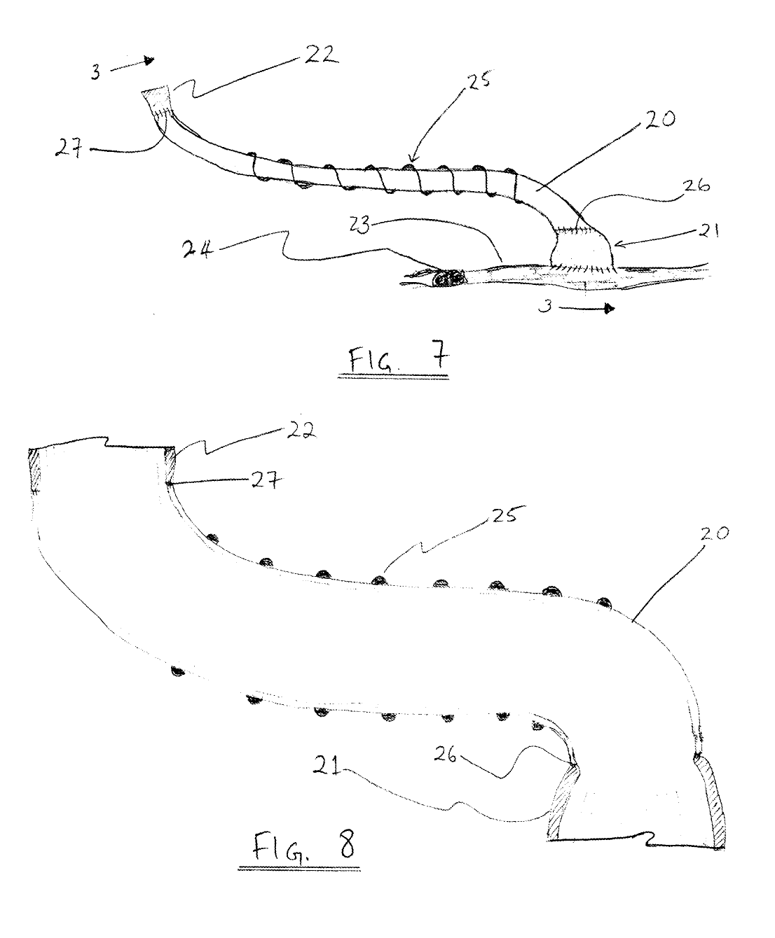

FIG. 1 is longitudinal cross sectional view of an exemplary stent graft or intra-luminally supported graft in accordance with an embodiment of the present invention. FIG. 2 is an expanded three dimensional view of the stent graft of FIG. 1. FIG. 3 is a radial cross sectional view of the stent graft of FIG. 1 compressed into a delivery catheter.

A stent graft is a device that is used when it is necessary to prop the lumen of a blood vessel open, especially when it is partially occluded with plaque, or the fatty deposits that can cause heart attacks. The use of a metallic stent is popular because this allows “minimally invasive surgery”, i.e. the compression of the stent-graft and insertion of said compressed stent-graft into the delivery catheter, and the use of such a catheter to enter the body through a blood vessel, to track the stent into place, and deployment of the stent without the need for surgery.

In FIG. 1 an exemplary stent-graft is shown in the expanded form. Stent 3 is laminated between polytetrafluoroethylene (PTFE)₂, or other suitable material, and treated tissue 1. Note that other fluoropolymers and other synthetic biomaterials may also be used, for example fluorinated ethylene propylene (FEP), aqueous dispersion PTFE, polyesters, nylons, polyethylene glycol, polyurethanes, silicones and siloxanes, and non-synthetic biomaterials such as alginates, cellulose etc. Other suitable materials that may be used to laminate the stent onto the tissue will be readily apparent to those skilled in the art in light of the teachings of the present invention.

In the present embodiment, the tissue is treated according to the invention WO/2006/026325 by Pathak et al. and placed on the luminal or internal stent surface. Although this is preferred, note that other methods of processing tissue may also be use as described above in the issued patents to Pathak, Freeman and Francis, to mention but a few. Note that tissue that is mainly collagen (devoid of its outer cellular layer) may be used instead. If the application requires some longitudinal elasticity, a tissue that has a large proportion of elastin may be selected. Ideal tissue thickness would be 0.05-2 mm but may differ depending on the application.

The PTFE is extremely thin, and may be in the range of 0.05 to 1 mm in thickness and possess a porosity characterized by a 10-60 micron internodal distance. It is placed on the abluminal or external stent surface, thus creating a stent “sandwich” between the two materials. The laminating materials are kept in place on either side of the stent by using polymeric attachments 4 between the tissue 1 and the PTFE 2. These attachments are primarily through openings between the struts of the stent 3. The PTFE 2 on the abluminal stent surface is designed with a low coefficient of friction as is characteristic of polytetrafluoroethylene. FIG. 2 shows the sandwiched stent structure 6. The stent graft is tubular with PTFE 2 on the abluminal or external surface and the treated tissue 1 on the luminal surface. Alternatively, the two materials may be sutured or stapled together between stent struts, they may be woven into each other if strips are used, or both materials may be bonded to the stent structure itself rather to each other. Polymeric attachments may be PEO, but also may be other adhesives such as polyethylene glycol (PEG), polyurethanes, tissue glues, polymethyl methacrylates (PMMA), etc. Note that pharmaceutical compounds may be mixed in with the adhesives, attached to the tissue directly, or may be infused into the pores or interstices of the PTFE itself using a carrier whose degradation or subsequent release of the drug is triggered by light, UV radiation, heat, or contact with a catalyst to name but a few techniques.

Typically stent grafts are taken and compressed down, eliminating most of their lumen, in order to be inserted into the much smaller delivery catheter. In general, the smaller the catheter is in diameter the easier for it to be inserted and tracked into the blood vessel for placement. A radial cross section of the compressed stent-graft inside the delivery catheter is illustrated by way of example in FIG. 3. The external PTFE surface 2 would serve as a lubricious interface between the compressed stent graft and the delivery catheter inside surface 8, allowing for easier loading and deployment of the stent graft. Note that other materials that possess a lubricious surface may also be used. Examples are nylons, polymers such as polyvinyl chloride (PVC) polyether block copolymers (e.g., Pebax,®), polyolefins (e.g., Hytrel®, DuPont, Wilmington, Del.), and the like, can be employed. Other possible polymers include polyethylene, polypropylene, polystyrene, polyethylene terephthalate, polyesters, silicone and polymers such as polyfluorocarbons or polysulfones. In additional other materials coated with a lubricious coating such as a hydrophilic polymer may also work well for this application.

Typically, stents and stent grafts are delivered over guide wires, not shown. Guide wires are the “rails” over which the delivery catheter is run. These springy and flexible rails can be seen with fluoroscopy, x-ray, as the physician guides it through the vasculature into the correct part of the blood vessel to be treated. Once the guide wire is in place, “over-the-wire” delivery catheters are run over the guide wire, running over and through lumen 9, until the stent graft is in position. Once in place, the delivery catheter is pulled back over the stent graft, allowing the spring steel of the stent, or temperature response of, for example, but not limited to, Nitinol to force the stent-graft to expand, thereby opening up into the blood vessel, but allowing blood to continue flowing through its lumen once the delivery catheter is withdrawn. Nitinol, or nickel-titanium alloy, is a metal with shape memory. That is, it remembers what shape it was formed into, and then under cold temperatures, can be formed into other shapes with ease. Once returned to its trained temperature, it reverts back to its pre-programmed shape, thus the name “shape memory” This alloy has an Austenite phase and a Martensite phase, cooling the stent to a temperature below the Martensitic transformation temperature (temperature induced Martensite) allows physical manipulation. This material is ideal for a stent as it can be “trained” at the dilated or expanded shape, chilled and compressed to be loaded into the delivery catheter, and then when it sees body temperature without the delivery catheter to constrain it, it will expand back to it's pre-programmed shape thereby supporting or propping open the blood vessel to be treated. One such patent that describes this material in stent form is U.S. Pat. No. 6,042,606 to Frantzen. Note that other stent materials for this application include superelastic Nitinol (stress-induced Martensite), stainless spring-steel, titanium, alloys, coated Nitinol etc. Even plastics may be used here, especially those that can be produced by dialing in the degradation rate to render the stent temporary.

Referring back to FIG. 1, the tissue luminal surface 1 would not create a rejection response to blood components passing through the lumen 5 and would harness the ability to endothelialize (grow endothelial cells which protect the surface,) and heal. In addition, a drug-loaded polymer can be attached to the tissue and then dissolved or delivered into the blood stream over time in order to enhance/accelerate the healing response. The objective of creating a healed surface is that the endothelial cells present create and emit the body's natural biochemicals (such as nitric oxide) thus preventing the blood components from attacking it. Other alternatives to the healed surface is complete passivation whereby an protein amino acid layer (e.g. albumin) is laid down, once again deterring blood components from trying to remove or inactivate the foreign material. Other methods used to enhance and accelerate healing include seeding the surface with endothelial cells prior to implant, seeding the surface with cells derived from bone-marrow, using growth hormones such as vascular endothelial growth factor (VEGF) that will grow capillaries and give rise to endogenous endothelial cells, growing a matrix or layer of live tissue on the surface prior to implant in the lab, etc. Other techniques to enhance/accelerate the healing response will be readily apparent to those skilled in the art in light of the teachings of the present invention.

The tissue 1 can be selected from intestinal mucosa/membrane or omentum, for example, or other tissues that are typically very thin, in order to maintain a low profile and the PTFE, for example, can be manufactured very thin and strong in order to complement the tissue in the area of strength. As mentioned earlier, the smaller the diameter of the stent graft and subsequently the delivery catheter, the easier it is to perform the treatment and navigate the delivery catheter through a tortuous vasculature. Furthermore, a selection of a tissue source with a high coefficient of radial expansion will allow the use of balloon-expandable stent applications such as the use of an angioplasty balloon to assist the stent structure to expand radially. The external PTFE can be designed to radially dilate along with the tissue and stent. For such a configuration, it will be necessary to have a deflated angioplasty balloon positioned in the lumen of the stent graft, and then for the physician to inflate the balloon once the delivery catheter has been pulled back. This will force the stent graft to expand in the radial direction, thus allowing the structure to become tubular, and sit with snug or close “apposition” within the blood vessel walls. Such technology was originally described in U.S. Pat. No. 4,733,665 to Palmaz. Although the biomaterial mentioned in this preferred embodiment is PTFE, other synthetic biomaterials may also be used in the same fashion. The combination synthetic biomaterial with the treated tissue component mitigates deficiencies found in current devices. In another embodiment of the present invention using shape-memory treated tissue as taught by Pathak et al. a tissue stent is used in conjunction with PTFE as a stent graft and the metallic stent component is omitted. The tissue based stent can be configured to be biostable, or partially or completely degradable in accordance with known techniques. Tailoring the degradation process will depend on the clinical application involved, e.g. a coronary artery might need stent support for only 2-4 months after implant. Pathak et. al. describes how to dial in the degradation rate, but alternate methods of processing tissue may adjust the crosslinking step, for example, so that the degradation rate can be controlled.

FIG. 4 is a perspective view of an exemplary vascular patch in accordance with an embodiment of the present invention. A treated tissue 10 and PTFE 11 are configured to be used as a vascular patch for artery patching, fistula intervention, pericardium repair etc. The patch is sutured into place, as such, for typical applications, the material would have to be thin and supple enough to pass a fairly small needle and suture. It is also easy to trim to size/shape with scissors so that the surgeon can tailor the product to the application. The patch would be sutured to the tissue opening or hole, just as one would patch a hole in trousers. If used on the heart or a blood vessel, the tissue 11 would be the blood contact surface, and the PTFE 10, or other biomaterial, would be the outer surface supplying the strength and resistance to suture hole bleeding, which is a problem with synthetic patches. In the situation where additional resistance to suture-hole bleeding is required, additional polymer can be used at the tissue-PTFE interface, or polyurethane with self-sealing properties. This polymer should have the ability to seal around the suture (conform to the shape of the suture, just as water surrounds a finger that is inserted into it) as it goes in and out of the patch, thus preventing bleeding from the suture line. By surrounding the suture circumference, blood is prevented from seeping out between the suture and the hole made by the suture needle. In another embodiment, the PTFE layer is made very thin and strong and the tissue surface is loaded with drugs that can be dissolved or delivered into the blood stream to enhance/accelerate healing and cellular incorporation. Applicable drugs are, for example but not limited to, anti-restenosis agents, anticoagulants, anti-infective compounds, growth factors, and other synthetic or biological compounds.

FIG. 5 depicts an exemplary arterio-venous (AV) access graft in accordance with an embodiment of the present invention. In a patient with kidney failure, the toxins in the blood need to be cleaned out externally as the kidneys normally provide this function. Passing the blood through a dialysis machine with a suitable filter that removes the toxins and then returning the cleaned blood to the body accomplish this. A shunt allows the dialysis machine access to a blood flow in the region of 0.4 to 1 liter of blood per minute, thus keeping the dialysis session down to 2-3 hours several times per week. AV or arterio-venous shunts/grafts are surgically placed into the body under the skin typically between a vein and an artery to create a path of rapidly flowing blood. The shunt is placed by tunneling it under the skin, then creating a suture line 16 between a source artery 15 and outflow vein 14, sutured in place at 17. The hemocompatible tissue surface 12 would be on the luminal, inside surface attached to abluminal, external, PTFE 13 using PEO or polyethylene glycol (PEG) or other suitable material as the adhesive. The lubricious outer surface of the PTFE is low friction and allows for ease in tunneling the graft under the skin. The PTFE 13 also bolsters the strength of the tissue, preventing any chance of dissection or blowout. Furthermore, in alternative embodiments, the tissue is configured to deliver drugs that dissolve into the blood stream to enhance/accelerate healing. Another embodiment of the AV access grant features a cannulation region. As with most PTFE AV grafts, a ten day to two week maturation period is required after implant or creating the shunt, before it can be used for dialysis. The needles used to remove and return the blood into the graft are fairly large, and leave behind gaping holes that often take a while to clot over and stop bleeding once the dialysis session is completed. Leaving the graft under the skin for two weeks before using it for dialysis allows some level of cells to grow into the graft surface, thereby enabling the holes left behind by the needles to close over more rapidly. Creating a cannulation, or needle entry region can circumvent this two week maturation time, especially if the implant surgery is done well with little swelling and inflammation. Other ways to circumvent or shorten this 2 week maturation time include using compounds to accelerate cellular infiltration into the PTFE surface such as collagen or gelatin, polyester particles, etc. A healthy incorporation of cells into the external surface of the graft will help the hole left behind by the needle to close spontaneously. Other techniques to circumvent this two week maturation time will be readily apparent to those skilled in the art in light of the teachings of the present invention.

FIG. 6 illustrates the exemplary use of a space between the tissue and biomaterial surfaces to create a cannulation region for the AV access shunt in FIG. 5. A sealant 18, for example but not limited to silicon rubber, can be trapped here to serve as an early cannulation region for immediate dialysis access using a dialysis needle 19, as described in WIPO patent application #WO/2006/026725 to Edwin et al. Once a needle is pulled out of the silicon cannulation region, the silicon will seal over the hole, just like a vaccine vial when the syringe needle is pulled out. In a further embodiment of the invention, the sealant can also be treated with a clot promoting drug or material for ease of use in dialysis access. This clot promoting drug would cause quick clotting of the blood that tried to exit through the needle hole. One example, but not limited to, of such a drug is thrombin. One example, without limitation, of a clot promoting material is polyester. In another embodiment, the PTFE 13 can also be porous but with some or all pores filled with a material such as gelatin. Gelatin is known for its ability to attract cells and to encourage tissue incorporation external to the graft. This is another mode of assisting bleeding cessation after dialysis needle withdrawal as this encourages tissue incorporation that then helps to close up the needle tract or hole, left behind by the large dialysis needle. The use of a graft material with some elasticity (e.g. the Vectra graft (Thoratec, Calif.) which is made out of porous and non-porous polyurethane, will give the graft wall inherent self-sealing properties to force bleeding cessation and therefore will allow early cannulation. Other techniques to assisting bleeding cessation after dialysis needle withdrawal will be readily apparent to those skilled in the art in light of the teachings of the present invention.

FIG. 7 illustrates an exemplary bypass graft in accordance with an embodiment of the present invention. In patients with occluded or badly diseased blood vessels, sometimes the only way to get blood flow past the blockage to the extremities would be to jump around it, or “bypass” the occlusion much as one would detour around a road construction site. This would send blood to areas that are not receiving it due to the blockage, thus relieving the “ischemia” or dearth of blood symptoms. It is well documented in the clinical literature that peripheral PTFE bypass grafts fail due to intimal hyperplasia or prolific cell growth at the ends, typically the distal end, of the graft. The PTFE material creates a slow rejection response that eventually occludes or shuts down the flow of blood either into or out of the graft. However, using tissue as the transition from graft to blood vessel can circumvent this. FIG. 7 shows by way of example how an improved device is constructed in accordance with an embodiment of the present invention. PTFE 20 is used as a bypass graft configured with the treated tissue attached at the distal end 21 via a suture line 26 or other attachment means. In a further embodiment of the present invention, the same can be done at the proximal end as well by suturing a band of tissue 22 via a suture line 27 or other attachment means to the PTFE. The use of this technique distally simulates the Taylor patch or Miller cuff configurations utilized to enhance the patency of a peripherally placed PTFE graft used for infrainguinal bypass, as described in published article “Reduced Elastic Mismatch Achieved by Interposing Vein Cuff in Expanded PTFE Femoral Bypass Decreases Intimal Hyperplasia” found in Artificial Organs, Volume 29, 2005 by Edmundo I., et. al. This technology is also recognized and sited in references in U.S. Pat. No. 6,589,278 to Peter Harris and Thien How. It should be noted however that one of the reasons this technique is rarely used in the US is because of the time and difficulty involved to harvest the vein from the patient. Using properly treated animal tissue (e.g., as described by Pathak et al.) can solve this issue and allow the device to come pre-packaged, assembled, and ready for use. This particular embodiment could be used in several different configurations and shapes to construct Miller Cuffs, Taylor Patches, or St. Mary Boots used to enhance the patency of grafts sutured to a below knee outflow artery 23 bypassing an occlusion 24. Alternatively in another embodiment, the tissue can be used as the luminal surface throughout, obviating the need for a suture line or attachment to the PTFE, as illustrated in the AV graft of FIG. 5. The PTFE or other synthetic biomaterial could be used as the reinforcing abluminal surface for ease in tunneling, due to its low coefficient of friction, and if reinforced with spiral or ringed beading 25, can also offer kink and crush resistance to the graft as is typical of several brands of PTFE grafts. This will bolster the vessels strength, preventing dissection or disruption. Alternatively, the hybrid device can be designed with circumferential ridges, as in an accordion, which would also serve to increase kink resistance.

FIG. 8 depicts a longitudinal cross sectional view of the bypass graft of FIG. 7. Although tissue is attached only at the proximal and distal ends of the graft in this illustration, as mentioned above, a further embodiment has the tissue line the entire inside surface and is laminated to the PTFE outer tube with the use of polymeric adhesives.

A further embodiment of the bypass graft described above is a continuous luminal tissue layer attached to an outer reinforcing layer, as depicted in the AV graft configuration, but a graft that would serve as a coronary bypass graft, obviating the need for harvesting saphenous vein or usage of the internal mammary artery as is typically done now for multiple vessel bypasses.

Other embodiments include synthetic-biomaterial degradable-tissue composites, as taught by Pathak et al. The tissue is configured to partially or completely degrade over time leaving behind a PTFE surface either indigenous or with a pharmaceutical or growth factor such as Endothelial Cell Progenitor (ECP) compound or a Vascular Endothelial Growth Factor (VEGF) attached to force endothelialization or healing/incorporation of this surface.

Another embodiment uses a highly porous but longitudinally compressed PTFE in conjunction with crimped tissue for enhanced kink-resistance. The graft material is formed into an accordion-like tube to allow for enhanced bend radii.

Another embodiment of the present invention uses the treated tissue-PTFE hybrid for a wound dressing. The tissue provides the biocompatible interface with the body's own tissue, and the PTFE protects it from the external environment. The hybrid can also be configured to remain permanently or can be configured to peel away or detach the PTFE when healing is complete and the tissue portion has degraded.

Yet another embodiment of the present invention uses the treated tissue-PTFE hybrid as a hernia patch. In patients with hernias, i.e. a protrusion of the abdominal contents or bowels, often a patch must be placed between the bowel and the abdominal muscles. Typically such materials are required to allow for adhesions or cellular incorporation external to the abdominal cavity so that healing can occur to the muscle bed, but be adhesion-free on the surface that is in contact with the abdominal viscera or bowels so that these can move freely, as is required during the digestion process. This is an ideal application for the hybrid product. PTFE or another synthetic polymer is rendered non porous and slick in order to resist adhesions to the viscera simply by spraying a biocompatible polyurethane or other soluble polymer onto the surface. This polymer fills the pores and render the surface non-porous and slippery. The tissue is attached to the other side of this layer, and faces the muscle bed allowing for adhesions, incorporation and healing. Note that as mentioned above, it is advantageous to avoid major surgery. In other embodiments, this device may also be designed to be minimally invasive by using an internal collapsible support structure that can be collapsed into a catheter, positioned over the hernia site, then deployed in place.

Another embodiment of the present invention uses the treated tissue-PTFE hybrid for a heart valve. A metallic or other solid support structure is used for the leaflet, with a synthetic polymer as the interface between the leaflet and the tissue cover. Both sides of the heart valve are the treated tissue with the PTFE and leaflet sandwiched inside. The PTFE offers the strength and durability required for the constant movement of the valve whereas the tissue offers the biocompatibility required for the blood contact surface.

Another embodiment of the present invention uses the treated tissue-PTFE hybrid for an abdominal aortic aneurysm (AAA) stent graft. Unlike the stent graft embodiment mentioned previously, an AAA stent graft is used primarily to exclude or repair an aneurysm, which is the weakening and subsequent dilation or abnormal stretching of a blood vessel such as the abdominal aortic artery. In patients that have such aneurysms, the mortality rate is more than 90% if the aneurysm ruptures, therefore if diagnosed, treatment is imperative. Standard surgical repair is possible by implanting a graft to replace the aneurysm. This type of surgery is extensive and many are not candidates for such major surgery. Thus patients and physicians may prefer the minimally invasive approach or the use of a stent graft as mentioned previously. Construction of this stent graft has treated tissue as the luminal blood contact surface and PTFE as the external or catheter contact surface, and a solid stent structure in between. An additional interface of tissue glue at the proximal and distal necks of the stent graft reduce or eliminate the current problem with endo-leaks which often occur due to a poor seal at the stent-graft to artery transition. This is usually due to the lack of good sealing or apposition between the host artery and the stent-graft. Tissue glues such as, but not limited to, Focalseal marketed by Genzyme Biosurgery or Duraseal marketed by Confluent surgical or simple methacrylates are candidates.

Another embodiment of the present invention uses the treated tissue-PTFE hybrid for a closure device. As mentioned earlier, minimally invasive surgery is now preferred to open surgery due to the reduced surgery, recovery time, and morbidity. However there is an unmet need for an efficacious closure device to be used at the entry site for the delivery catheter. That is, the hole in the blood vessel, surrounding tissue and external skin that remains when the catheter and guide wire are withdrawn. If the catheter is large in diameter, this hole will also be large and may take a long time to stop bleeding. The invention described here can be used to design a plug for such a hole. Devices that perform this function are already on the market. A treated tissue-PTFE hybrid device design, in accordance with the present invention, provides the hemocompatibility of the tissue for the internal blood vessel and the synthetic polymer such as, but not limited to, PTFE provides the strength at the external surface outside of the blood vessel and would also be incorporated by the tissue and muscle bed. The PTFE is used to fill the hole in the tissue bed, and could also be designed to incorporate the delivery method.

Another embodiment of the present invention uses the treated tissue-PTFE hybrid for a septal defect repair device. This is often a congenital condition wherein there is an unwanted communication or unnatural opening from one chamber of the heart into another. When diagnosed, this defect requires major invasive surgery when the patient is sometimes still a baby. Therefore it is desirable to have a device that can be placed using minimally invasive technology. A device like two pin wheels back to back, one on either side of the defect is delivered using a catheter. As with the heart valve, treated tissue is used on the “pin wheels” on either side of the defect as the blood contact surfaces, with the stent like structure and synthetic biomaterial sandwiched inside. The stent structure is a shape memory material such as, but not limited to, Nitinol which will spring into its pre-programmed shape when deployed from the delivery catheter. Alternatively, the device is configured for standard surgical placement.

Another embodiment of the present invention uses the treated tissue-PTFE hybrid for a central line, or AV catheter. Such catheters are inserted into the neck region typically into the internal jugular vein, and then pushed into the right atrium of the heart. These catheters can be used for dialysis as long as this is on a temporary basis. By affixing tissue to the outside of a catheter, the tissue can be configured to deliver drug compounds that would resist fibrin sheath formation and clotting that plague current AV catheters. If used as the cuff component at the site of catheter entry, a reduction in infection would result as the tissue would heal and fuse with the patients' skin and tissue bed.

Other embodiments in accordance with the present invention apply to orthopedic applications where harvested and processed tissue is bonded and wrapped around one or more synthetic biomaterials to bolster the tissue's strength and durability. Replacements or repair of ligaments, muscle and tendons are potential candidates for this technology. In these situations when tremendous strength and durability is required from the implant, the tissue is externalized, whereas the PTFE or other biomaterial is internalized to provide the strength for the application.

Other embodiments of the present invention include all the embodiments mentioned above, but with the addition of radiopaque compounds incorporated into the treated tissue along with the processing polymer as mentioned in Pathak et al. This assists the physician in identifying the implant fluoroscopically, or under x-ray.

It should be recognized that although expanded PTFE has been cited as the synthetic biomaterial of choice, this invention does not limit itself to this polymer. Other examples of synthetic biomaterials include, but are not limited to, polyurethane, polyethylene, silicone rubbers, polyesters, nylon etc. PTFE is preferred because it is chemically inert and can be made with the required physical properties for this application. However, alternate biomaterials can also be used although each will have its own deficiencies. If polyurethane is used, its creep resistance and hemocompatibility should be addressed and modified. If silicone is used, it should be made porous and strong while maintaining a low profile and good kink resistance. If polyester is used, its inflammatory response and strength need to be addressed. Also, all these alternate materials should be rendered lubricious for ease in loading and deployment from the delivery catheter, as well as in tunneling for the AV graft application. In addition, if alternate materials are used, there should be some way of ensuring that the bond between the tissue and the biomaterial is sufficiently strong and durable for the application.

Nor should only synthetic biomaterials be candidates for attachment to the tissue. Biological biomaterials such as, but not limited to, seaweed and chitin extracts are examples of other potential candidates.

It should also be recognized that although animal tissue of bovine, ovine, and porcine origin have been mentioned, all biological tissues including that of human origin can also be used and treated accordingly.

It should furthermore be recognized that although the technology of Pathak and Thigle has been cited, this in no way limits the invention to tissue processed in this way. Other methods of processing including glutaraldehyde techniques may still be employed as the combination of the two materials will still be a substantial improvement over either one alone. As mentioned earlier, other methods of treating animal tissue may also be acceptable, such as described by Freeman et al. in U.S. Pat. No. 4,798,611. The use of glutaraldehyde to crosslink the tissue is described here as well as gamma sterilization to reduce immunogenicity and to enhance handling characteristics. Yet another approach to the treatment of tissue for clinical use is described in U.S. Pat. No. 6,132,986 to Pathak et. al. where tissues are exposed to di or polyfunctional acids. Another tissue preparation that may be used if biodegradable tissue is desired as described in U.S. Pat. No. 6,652,594 to Francis et al. in which the material is treated by alkylating its primary amine groups to reduce antigenicity, but permitting its use in-vivo without crosslinking Pathak et al. also describes a non-glutaraldehyde crosslinking technique in U.S. Pat. No. 6,596,471 using a bis-maleimide compound. Appropriate treatment of tissue in this context refers to tissue processed in such a way that it retains its strength, natural feel and handling properties; is not cytotoxic or inflammatory, and is biostable or with controllable degradable properties.

It should also be reiterated that attachment of the treated tissue to synthetic biomaterial may be accomplished in a variety of ways such as, but not limited to, using polymeric materials that are chemically bonded to tissue but mechanically bonded to synthetic biomaterial by heat and pressure, as well as more simplistic but durable methods such as suture or adhesives, or simply using strips of both and weaving them together.

Note also that in several of the embodiments mentioned, multiple layers of PTFE and tissue may be used rather than just one. Depending on the application, PTFE may be sandwiched between two layers of tissue, tissue may be sandwiched between two layers of PTFE, or multiple layers of either one or both could be used.

The biomaterial in use could also be plastic or even metallic. One example would be in the case of a vena cava filter. This device is placed in the vena cava prior to the right atrium to break up any blood clots before they reach the lung. Bonding or coating the vena cava with tissue to render it biocompatible is within the scope of the current invention.

As is bonding or coating PTFE, metal, or plastic with tissue prior to the hybrid medical device being constructed, as in the case of a custom device whereby the physician may want to configure the device himself prior to implant, or the raw material may be sold as an OEM product.

Other applications/embodiments include suture, wound dressings, dura-mater substitutes, penile implants, cheek implants for cosmetic surgery, etc. etc.

In the embodiments, animal tissue is mentioned, however it must be reiterated that although rare and expensive, tissue of human origin may also be used. As might animal tissue manufactured from genetically modified animals. In fact with this material, less processing may be required as the tissue may already be rendered non-reactive. Tissue grown in the laboratory is another source, as this may also be non-reactive.

The embodiments cover layers of synthetic biomaterial, however strips or alternating bands may also be considered, as would be the same for tissue.

Although the embodiments refer to tissue, note that this encompasses leather as well as leather with hair and/or fur attached.

Having fully described at least one embodiment of the present invention, other equivalent or alternative treated tissue-PTFE hybrid compositions will be apparent to those skilled in the art. The invention has been described above by way of illustration, and the specific embodiments disclosed are not intended to limit the invention to the particular forms disclosed. The invention is thus to cover all modifications, equivalents, and alternatives falling with the spirit and scope of the following claims.

What is claimed is:

1. A hybrid medical device comprising:
at least one synthetic biomaterial; and
at least one treated biological tissue suitable for implantation attached to said biomaterial wherein said tissue provides a blood contact surface and said biomaterial provides structural support.

2. The device as recited in claim 1, wherein said tissue and biomaterial are attached using a polymer.

3. The device as recited in claim 2, wherein said polymer is chemically or mechanically attached to said tissue.

4. The device as recited in claim 1, further comprising pharmaceutical compounds for delivery over time.

5. The device as recited in claim 1, further comprising radiopaque compounds for fluoroscopic visualization.

6. The device as recited in claim 1, wherein said tissue degrades over time.

7. The device as recited in claim 1, wherein said tissue only partially degrades over time.

8. The device as recited in claim 1, wherein said biomaterial comprises polytetrafluoroethylene (PTFE).

9. The device as recited in claim 1, wherein said device is configured for use as a heart valve.

10. A stent graft for opening a lumen of a blood vessel, the stent graft comprising:
a stent support structure comprising struts, an internal surface and an external surface;
at least one treated biological tissue suitable for implantation disposed on said internal surface to provide a luminal surface for the blood vessel; and
at least one synthetic biomaterial disposed on said external surface wherein said tissue and said biomaterial are attached through openings between said struts and the stent graft is compressible for delivery into the blood vessel via a catheter.

11. The stent graft as recited in claim 10, wherein said stent support structure comprises shape memory properties.

12. The stent graft as recited in claim 11, wherein said stent support structure comprises treated biological tissue.

13. The stent graft as recited in claim 10, wherein said stent support structure comprises metal.

14. The stent graft as recited in claim 10, wherein said stent support structure comprises plastic.

15. The stent graft as recited in claim 10, wherein said synthetic biomaterial comprises a low friction lubricious material for ease in loading and deployment from the catheter.

16. The stent graft as recited in claim 10, wherein said tissue material is biostable and supports endothelialization and healing.

17. The stent graft as recited in claim 10, further comprising radiopaque materials or markers.

18. The stent graft as recited in claim 10, wherein said tissue and biomaterial are attached using a polymer.

19. The stent graft as recited in claim 10, further comprising pharmaceutical compounds for delivery over time.

20. The stent graft as recited in claim 10, wherein said tissue comprises a high coefficient of radial expansion allowing the use of an angioplasty balloon to assist the stent graft to expand radially.

21. The stent graft as recited in claim 10, wherein said biomaterial comprises polytetrafluoroethylene (PTFE).

22. A vascular patch comprising:
a synthetic biomaterial; and
a treated biological tissue suitable for implantation attached to said biomaterial wherein said tissue provides a blood contact surface, said biomaterial provides structural support, the patch is suitable for suturing and the patch can be trimmed to fit.

23. The vascular patch as recited in claim 22, wherein said tissue is attached to said biomaterial using a polymer.

24. The vascular patch as recited in claim 23, wherein said polymer provides additional resistance to suture-hole bleeding.

25. The vascular patch as recited in claim 22, further comprising pharmaceutical compounds.

26. The vascular patch as recited in claim 22, wherein said biomaterial comprises polytetrafluoroethylene (PTFE).

27. An arterio-venous (AV) access graft comprising:
a continuous treated biological tissue suitable for implantation forming a luminal blood-contact layer; and
a synthetic biomaterial attached to said tissue forming an abluminal layer providing structural support to said tissue when the graft is sutured between a vein and an artery.

28. The AV access graft as recited in claim 27, further comprising a cannulation region between said tissue and said biomaterial.

29. The AV access graft as recited in claim 28, further comprising sealant disposed in said cannulation region for sealing a hole produced by a dialysis needle.

30. The AV access graft as recited in claim 29, wherein said sealant comprises pharmaceutical compounds.

31. The AV access graft as recited in claim 27, wherein said biomaterial is porous and at least some of said pores are filled with a gelatin material to encourage tissue incorporation.

32. The AV access graft as recited in claim 27, wherein said biomaterial is lubricious for ease in tunneling.

33. The AV access graft as recited in claim 27, wherein said biomaterial comprises polytetrafluoroethylene (PTFE).

34. A bypass graft comprising:
a tubular synthetic biomaterial of suitable dimensions for use in bypassing a blood vessel; and
a treated biological tissue suitable for implantation attached to a distal end of said biomaterial wherein said tissue mitigates effects of intimal hyperplasia.

35. The bypass graft as recited in claim 34, further comprising treated biological tissue suitable for implantation attached to a proximal end of said biomaterial wherein said tissue mitigates effects of intimal hyperplasia.

36. The bypass graft as recited in claim 35, further comprising spiral or ringed beading on the abluminal surface of said biomaterial to provide kink and crush resistance.

37. The bypass graft as recited in claim 35, further comprising treated biological tissue suitable for implantation used as the luminal surface of the graft.

38. The bypass graft as recited in claim 35, wherein said tissue is attached at said distal and proximal ends by suturing.

39. The bypass graft as recited in claim 34, wherein said biomaterial comprises polytetrafluoroethylene (PTFE).

40. A hybrid medical device comprising:
means for providing at least one synthetic biomaterial;
means for providing at least one treated biological tissue suitable for implantation; and
means for attaching biomaterial to said tissue wherein said tissue provides a blood contact surface and said biomaterial provides structural support.

41. The hybrid medical device as recited in claim 40, further comprising means for providing pharmaceutical compounds.

42. The hybrid medical device as recited in claim 40, further comprising means for providing radiopaque compounds.
